(19) United States
(12) Patent Application Publication
(10) Pub. No.: US 2004/0058974 A1
(43) Pub. Date: Mar. 25, 2004

(54) Treating fungal infections

(75) Inventors: Rachel Courtney, Piscataway, NJ (US); Mark A. Laughlin, Woodbridge, NJ (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(73) Assignee: SCHERING CORPORATION

(21) Appl. No.: 10/667,856

(22) Filed: Sep. 22, 2003

Related U.S. Application Data
(60) Provisional application No. 60/412,985, filed on Sep. 23, 2002.

(51) Int. Cl. A61K 31/496; A61K 31/4178

(52) U.S. Cl. 514/397; 514/254.07

(57) ABSTRACT

Methods of treating or preventing fungal infections in humans of 12 years and older in need of such treating or preventing which comprises orally administering an effective amount of posaconazole in divided doses two to four times a day to produce an arithmetic mean steady state average maximum plasma concentration of posaconazole of at least about 300 ng/mL to at least about 500.

### BACKGROUND OF THE INVENTION

This invention relates to methods of treating or preventing fungal infections in humans of 12 years and older in need of such treating or preventing by orally administering an amount of posaconazole in divided doses two to four times a day effective to treat or prevent fungal infections

U.S. Pat. No. 5,661,151 discloses posaconazole and its use as antifungal agent with a broad spectrum of activity. In vitro and in vivo studies have demonstrated that posaconazole has good activity against Candida species (including albicans, glabrata, and tropicalis), as well as other opportunistic fungi, including but not limited to, Aspergillus, Fusarium, Basidiomycetes, Blastomyces, Coccidioides, Histoplasma, Zygomycetes, and Scedosporium, and opportunistic monilaceous and dematiaceous molds and dermatophytes.

Immunocompromised patients with human immunodeficiency virus (HIV) or acquired immunodeficiency syndrome (AIDS), as well as those patients undergoing bone marrow or solid organ transplant are at particularly high risk of developing serious fungal infections. The fungal infections most prevalent among immunocompromised patients include, but are not limited to, candidiasis, aspergillosis, cryptococcosis, and fusariosis. Despite the availability of amphotericin B and a number of newer antifungal therapies, morbidity and mortality from invasive fungal infections remain high. Posaconazole is a lipophilic drug and pharmacokinetic studies have shown that the oral bioavailability of posaconazole given in a single daily dose is increased approximately 4-fold when it is administered with a high fat meal compared to the fasted state. However, as immunocompromised patients often have poor oral intake, an evaluation of the pharmacokinetic profile of posaconazole under fasted conditions is required.

There is a current unmet medical need for the treatment of a wide variety of fungal infections in patients, especially invasive fungal infections in immunocompromised patients.

### SUMMARY OF THE INVENTION

We have discovered that the oral bioavailability of posaconazole is significantly increased in fasted subjects when posaconazole is orally administered in divided daily doses of 200 mg four times a day (“QID”) or 400 mg twice a day (“BID”)compared to 800 mg once a day administration. The average plasma concentrations of posaconazole achieved by such divided doses exceed the majority of the Minimium Inhibitory Concentrations needed to kill 90% (“MICs₉₀”) of the clinically relevant pathogenic fungi. Thus, this invention provides a method of treating and/or preventing fungal infections in immunocompromised patients who are at high risk of developing serious funal infections.

Thus, this invention provides a method of treating or preventing fungal infections in humans of 12 years and older in need of such treating or preventing which comprises orally administering an effective amount of posaconazole in divided doses two to four times a day to produce an arithmetic mean steady state average maximum plasma concentration of posaconazole that exceeds the majority of the Minimium Inhibitory Concentrations needed to kill 90% (“MICs₉₀”) of the clinically relevant pathogenic fungi.

This invention also provides a method of treating or preventing fungal infections in humans of 12 years and older in need of such treating or preventing which comprises orally administering an effective amount of posaconazole in divided doses two to four times a day to produce an arithmetic mean steady state average maximum plasma concentration of posaconazole of at least about 300 ng/mL to at least about 520 ng/mL.

This invention also provides a method of treating or preventing a fungal infection in humans of 12 years and older in need of such treating or preventing which comprises orally administering an effective amount of posaconazole in divided doses two to four times a day to produce an arithmetic mean steady state minimum plasma concentration (Cₘᵢₙ) of posaconazole of at least about 50 ng/mL at about 48 hours after the initial dose of the effective amount of posaconazole.

This invention also provides a method treating or preventing fungal infections in humans of 12 years and older in need of such treating or preventing which comprises orally administering about 200 mg of posaconazole four times a day to produce an arithmetic mean steady state average maximum plasma concentration of posaconazole of at least about 500 ng/mL to about 520 ng/mL.

This invention also provides a method treating or preventing fungal infections in a human of 12 years and older in need of such treating and/or preventing which comprises orally administering about 200 mg of posaconazole four times a day to produce a population mean area under the concentration-time (0-24 hr) curve of posaconazole of about 12 mcg.hr/mL.

This invention also provides a method treating or preventing fungal infections in a human of 12 years and older in need of such treating and /or preventing which comprises orally administering about 200 mg of posaconazole four times a day to produce an average plasma concentration of posaconazole of about 0.5 mcg.hr/mL.

This invention also provides a method treating or preventing fungal infections in a human of 12 years and older in need of such treating or preventing which comprises orally administering about 200 mg of posaconazole four times a day.

This invention also provides a method treating or preventing fungal infections in a human of 12 years and older in need of such treating or preventing which comprises orally administering a total dose of about 800 mg of posaconazole a day in three divided doses.

This invention also provides a method treating or preventing fungal infections in a human of 12 years and older in need of such treating or preventing which comprises orally administering about 400 mg of posaconazole two times a day.

### BRIEF DESCRIPTION OF THE FIGURES

FIG. 1 is a linear: linear graphic display of the mean posaconazole plasma concentrations (ng/mL) versus time (hours) following administration of oral posaconazole 800 mg as a single dose (-●-, regimen A), 400 mg, every 12 hrs, or BID (-Δ-, regime B), and 200 mg, every six hours or q 6 h, or QID (-□-, regimen C) to fasted subjects.

FIGS. 2A, 2B and 2C are linear: linear graphic displays of the observed plasma concentrations (ng/mL) versus fitted plasma concentrations (ng/mL) for posaconazole administered orally as a 800 mg single dose (FIG. 2A, regimen A), 400 mg BID (FIG. 2B, regimen B), and 200 mg QID (FIG. 2C, regimen C).

FIGS. 3A1, 3A2, & 3A3; FIGS. 3B1,3B2 & 3B3; and FIGS. 3C1,3C2 & 3C3 are linear: linear graphic displays of posaconazole plasma concentrations (ng/mL) profiles for three representative subjects following oral administration of posaconazole 800 mg as a single does (regimen A, FIGS. 3A1, 3A2, & 3A3), 400 mg “BID” (regimen B—FIGS. 3B1,3B2 & 3B3 ), and 200 mg “QID” (regimen C, FIGS. 3C1,3C2 & 3C3). The solid circles represent the observed plasma posaconazole concentrations (ng/mL) and the line represents the fitted profile. FIGS. 3A1, 3B1 and 3C1 are for a first subject. FIGS. 3A2, 3B2 and 3C2 display the posaconazole plasma concentrations for a second subject, and FIGS. 3A3, 3B3 and 3C3 display the posaconazole plasma concentrations for a third subject.

FIG. 4A is a linear: linear graphic display of the ratio of the population mean area under the concentration-time (0-24 hr) AUC(0-24) value for oral posaconazole regimen B (400 mg, BID) to the AUC (0-24 hr) value for posaconazole regimen A (800 mg, single dose QD) for each of the 18 healthy fasted subjects.

FIG. 4B is a linear: linear graphic display of the ratio of the population mean AUC (0-24hr) for oral posaconazole regimen C (200 mg, QID) to the AUC (0-24hr) value for regimen A (800 mg, single dose QD) to each of the 18 healthy fasted subjects.

FIG. 5 is a linear: linear graphic display of the steady state posaconazole plasma concentrations (ng/mL) versus time (hours) following administration of an oral suspension of posaconazole 200 mg once a day (QD)as a single dose (- -), 400 mg, once a day(QD) as a single dose (-▴-), and 200 mg, every six hours or q 6 h, or QID (-▪-) to neutropenic oncology (bone marrow transplant) patients with fluconazole-resistant Candida infections.

### DETAILED DESCRIPTION OF THE INVENTION

We have discovered that posaconazole oral bioavailabilty is significantly increased by administering posaconazole with divided daily dosing of 400 mg twice a day (“BID”, Regimen B) or every 12 hours and 200 mg four times a day (“QID”, Regimen C) or every 6 hours compared to orally administering 800 mg once a day (“QD”, Regimen A). The posaconazole bioavailability was determined to be statistically significantly different between regimens (P<0.001). Thus, the ratio of the bioavailability of Regimen B to Regimen A is 1.98±0.35 represently a 98% increase for 400 mg BID compared to 800 mg QD. The ratio of the bioavailability of Regimen C to Regimen A is 3.2±0.7, or a 220% increase for 200 mg QID compared to 800 mg QD. The average posaconazole plasma concentrations achieved by orally administering 200 mg of posaconazole using a BID and QID dosing regimen exceed the majority of the Minimum Inhibitory Concentrations needed to kill 90% (MICs₉₀) of the clinically relevant pathogenic fungi.

The methods of the present invention are also effective in treating or preventing fungal infections as well as opportunistic monilaceous and dematiaceous molds and dermatophytes in humans 12 years or older.

The term “fungal infections” as used herein means fungal infections caused by clinically relevant pathogenic fungi as well as refractory fungal infections and invasive fungal infections.

The term “clinically relevant pathogenic fungi” as used herein means opportunistic fungi, including but not limited to, Candida species (including albicans, glabrata, and tropicalis), Aspergillus, Fusarium, Basidiomycetes, Blastomyces, Coccidioides, Cryptococcus, Histoplasma, Microsporum, Trichophyton, Zygomycetes, and Scedosporium.

The term “invasive fungal infections” as used herein means serious fungal infections, especially fungal infections most prevalent among immuno-compromised patients.

The term “refractory fungal infections” as used herein means those fungal infections that are refractory or resistant to standard fungal therapies, including but not limited to, those therapies employing fluconazole, itraconazole or amphothericin B, including for example, amphothericin B liposomal formulations.

In accordance with the methods of the present invention, orally administering 200 mg of posaconazole four times a day or 400 mg of posaconazole twice a day to a human of 12 years and older with a fungal infection significantly increased the average posaconazole plasma concentrations compared to orally administering 800 mg of posaconazle once a day. Similar results are expected by orally administering 800 mg of posaconazole in divided doses three times a day, e.g., one dose 2×200 mg, and two 200 mg doses.

The term “an effective amount of posaconazole in divded doses two to four times a day” as used herein means 200 mg of oral posaconazole four times a day or 400 mg of oral posaconazole twice a day as well as 800 mg of oral posaconazole in divided doses three times a day, e.g. , one dose, 2×200 mg, and two 200 mg doses.

The average posaconazole plasma concentration produced by orally administering 400 mg of posaconazole twice a day (400 mg BID) is greater than about 300 ng/mL) and by orally administering 200 mg of posaconazole four times a day (200 mg QID) is greater than about 500 ng /mL.

The term “human of 12 years and older in need of treating or preventing fungal infections” includes immuno-comprised patients as well as any patient having a fungal infection susceptible to posaconazole.

The term “immunocomprised patient” as used herein includes oncology patients with neutropenia, e.g., neutropenic patients undergoing high dose chemotherapy, and/or bone marrow transplantion (“BMT”), as well as organ transplant recipient.

The term “susceptible fungal infection” as used herein includes, but is not limited to, candidiasis, aspergillosis, cryptococcosis and funsariosis as well as fungal infection due to Blasidiomycetes, Blastomyces, Coccidioides, Histoplasma, Zygomycetes, Microsporum, Trichophyton, and Scedosporium.

In accordance with the present invention, a human in need of treating or preventing a fungal infection, especially neutropenic patients having an invasive fungal infection or one refractory to other fungal agents, e.g., itraconazole, fluconazole or amphthericin B are administered 200 mg of oral posaconazole four times a day until the patient is considered stable by the attending clinician and thereafter 400 mg of oral posacaonazole BID until the fungal infection is eradicated as shown by standard testing procedures well known to those skilled in the art.

A proportion of our target therapeutic population has poor food intake, which can have a significant impact on oral bioavailability. This in combination with the dose-limited absorption may limit patients' exposure to posaconazole. Therefore, in an attempt to overcome the limited absorption, a clinical pharmacology study was performed to evaluate if the exposure to posaconazole in fasted subjects could be improved using divided dosing regimens.

The exposures in the fasted healthy volunteers were confirmed in a study of posaconazole pharmacokinetics in bone marrow transplant (BMT) patients. The results indicated that the exposure to posaconazole, given as 200 mg QID, was comparable between fasted healthy subjects (AUC˜12 μg·hr/mL) and BMT patients (AUC˜11 μg·hr/mL).

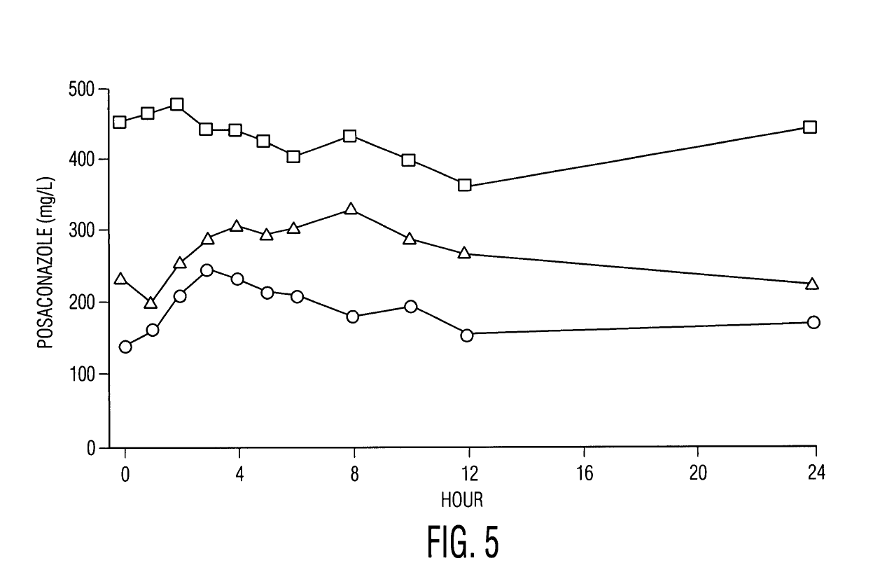

These results indicate that 200 mg of oral posaconazole QID for 7 to 30 days for acutely ill patients whose oral food intake may be erratic, is the most optimal dosage regimen in terms of reliable drug exposure for the treatment of severe or life-threatening invasive fungal infections (See FIG. 5).

Once a patient's oral food intake becomes stable on 200 mg of posaconazole QID, 400 mg BID is considered adequate to maintain the desired therapeutic concentrations for the treatment of invasive fungal infections. In addition, a reduction in dosing frequency, from QID to BID, in an outpatient setting is expected to aid in compliance thereby ensuring maintenance of steady-state concentrations. Following response to QID treatment, if a patient is deemed unstable on BID dosing, the prescribing physician may re-initiate 200 mg posaconazole QID.

In accordance with the present invention, a human in need of treating or preventing a fungal infection, especially neutropenic patients with an invasive fungal infection or one refractory to other fungal agents e.g., itraconazole, fluconazoles or amphthericin B will be administered 200 mg of oral posaconazole four times a day until the patient's fungal infection is considered stable by the attending clinician and thereafter 400 mg of oral posacaonazole BID until the fungal infection is eradicated as shown by standard testing procedures well known to those skilled in the art.

The term ‘stable” as used herein in reference to a patient's fungal infection means that there is no progression in the general signs or symptoms of the fungal infection or there is improvement in the relevant radiographic abnormalities as determined by the attending clinician. The duration of treating fungal infections with 800 mg of oral posaconazole in divided doses, two three or four times a day should be continued until the there is a complete clinical response. The duration may be up to six or even twelve months. The attending clinician will use discretion in determining the appropriate duration of the oral posaconazole therapy in accordance with the present invention based on evaluation of the following; the clinical diagnosis of the invasive fungal infection, the causative fungal pathogen, the severity of the invasive fungal infection, the severity of the patient's underlying disease, recovery from immunosuppression, and the rapidity of the clinical response as well as the age of the patient.

METHODS

Study Population

Men and women, 18 to 45 years of age were recruited for the clinical pharmacology study. Subjects were required to be of normal body weight (within 15% of ideal body weight) and generally in good health. Inclusion criteria included normal electrocardiogram (ECG) and laboratory test results, along with negative drug and pregnancy screens; nonlactating women of childbearing potential were required to use a barrier method of contraception during the study. Exclusion criteria included substance abuse or significant food or drug allergy, donation of blood or use of an investigational drug during the previous 90 days, use of any prescription or over-the-counter drugs other than acetaminophen in the previous 2 weeks, or use of alcohol during the previous 48 hours. Also excluded were subjects with a positive HIV, hepatitis B, or hepatitis C test, as were subjects with is a clinically significant systemic infection during the previous 4 weeks. Subjects who smoked ≧10 cigarettes daily were also excluded.

Study Design

This was an open-label, 3-way crossover study. Subjects who met the entry criteria were confined to the study center at least 12 hours prior to dosing and 48 hours after each dosing regimen. Subjects were discharged from the study site for a 1-week washout period between each arm of the study.

Following an overnight fast, each subject received posaconazole as an oral suspension (40 mg/mL), using one of 3 dosing regimens: a single dose of 800 mg (regimen A); 2 doses of 400 mg given 12 hours apart (regimen B); or 4 doses of 200 mg given 6 hours apart (regimen C). Subjects were randomly assigned using a computer-generated random code to 1 of 6 treatment schedules (ABC, ACB, BAC, BCA, CAB, CBA). The oral posaconazole suspension was manufactured and supplied by Schering-Plough Research Institute, Kenilworth, N.J., USA. After dosing with an oral dosing syringe, the oral cavity was inspected to ensure that the subject had swallowed the suspension. Subjects remained fasted for 24 hours after the initial dose. During the first 14 hours, water was provided ad libitum and subjects received IV 1,400 mL D₅W/0.5 normal saline at approximately 100 mL/hr as a calorie and fluid source.

For regimen A, B, and C, blood samples were collected in heparin-containing tubes at baseline (0 hr) and at 2, 4, 5, 6, 8, 10, 12, 16, 24, and 48 hours after the first dose. For regimen B, blood samples were additionally collected at 14, 17, 18, 20, and 22 hours after the first dose. For regimen C, additional blood samples were collected at 11, 14, 17, 18, 20, 22, 23, 26, 28, and 36 hours after the first dose. Each sample was centrifuged within 15 minutes after collection for 10 minutes at 4° C. and 1,500 g. The resultant plasma was frozen (−20° C.) until assayed. Plasma posaconazole concentrations were quantified using a validated high-performance liquid chromatographic (HPLC) assay with a lower limit of quantitation (LOQ) of 5 ng/mL and a linear range of 5.0 to 5000 ng/mL.

Safety

Physical examinations, vital signs, ECGs, and clinical laboratory tests were conducted at screening and at the end of the study (48 hours after the final dosing regimen). Treatment-emergent adverse events were recorded throughout the study. In addition, vital signs were evaluated prior to drug administration and at 24 and 48 hours after the first dose of each regimen.

Pharmacokinetic Analyses

The observed values were the maximum plasma concentration (Cₘₐₓ), time of maximum plasma concentration (Tₘₐₓ), and the trough plasma concentration at 48 hours after the initial dose of posaconazole (Cₘᵢₙ).

Based on previous experience in modeling concentration-time posaconazole data and goodness of fit criteria, a one-compartment oral model with a first-order rate of absorption and first-order rate of elimination was used. The intra-subject error was assumed to be additive, defined as,

C_{i,j}(t)=G_{i,j}(t)+ε_{i,j}(t)

where C_{i,j}(t) and G_{i,j}(t) were the measured and predicted concentrations, respectively, for the j^{th} subject at time t under the i^{th} regimen. The predicted concentrations G_{i,j}(t) following treatments B and C were calculated using the principle of superpositioning, with a lag time equal to the dosing interval (B: 12 hours; C: 6 hours). The error term ε_{i,j}(t) was assumed to be N(0, σ² _{ε}). Differences in exposure following the three dosage regimens were explored by allowing the bioavailability fraction (F) to differ between regimens. Using the bioavailability fraction of regimen A as a reference (F₁), which was fixed equal to 1, this allowed an estimation of the bioavailability fractions F₂ (regimen B) and F₃ (regimen C), relative to F₁. With these parameters, volume of distribution was estimated as V/F₁. Differences in the rate of absorption (Kaᵢ) or in the rate of elimination (Keᵢ) were also considered (i=1, 2, and 3, as above). Using individual parameter estimates, the area under the concentration-time curve from time 0 to infinity (AUC[I]) was determined for each subject.

The intersubject variability in each pharmacokinetic parameter, θ, was modeled as:

θⱼ=θ·e^{(η}_{_{j,θ}}⁾

where the random effect η_{j,θ} was assumed to be N(0, σ²_{,θ}), and was independent of other effects. Accordingly, the intersubject variability in F (combined with that of V) was expressed as η_{j, F}. As variability in bioavailability may also vary between regimens, we thus defined the random effects as:

F_{i,j}=Fᵢ. exp(η_{j,Fi})

with η_{j,Fi}˜N(0,σ²_{Fi}). Random effects were kept in the model only if they tested as significant.

The statistical significance of a variable was assessed using the likelihood ratio test at the 0.05 level of significance. The area under the plasma concentration-time curve was calculated using the model parameters and the individual (post-hoc) estimates. Modeling was carried out using the non-linear mixed effect (“NLME”) function of S-plus Version 6 (Insightful Corporation, Seattle, Wash.).

Results

Subject Demographics and Disposition

Eighteen men (13 Africian Americans, 5 Caucasians) enrolled and completed the study. Subjects ranged in age from 26 to 44 years (mean 36 years) and in body weight from 63.6 to 100 kg (mean 81.9 kg). All subjects who enrolled in the study completed the 3 dosing periods.

Observed Posaconazole Concentrations

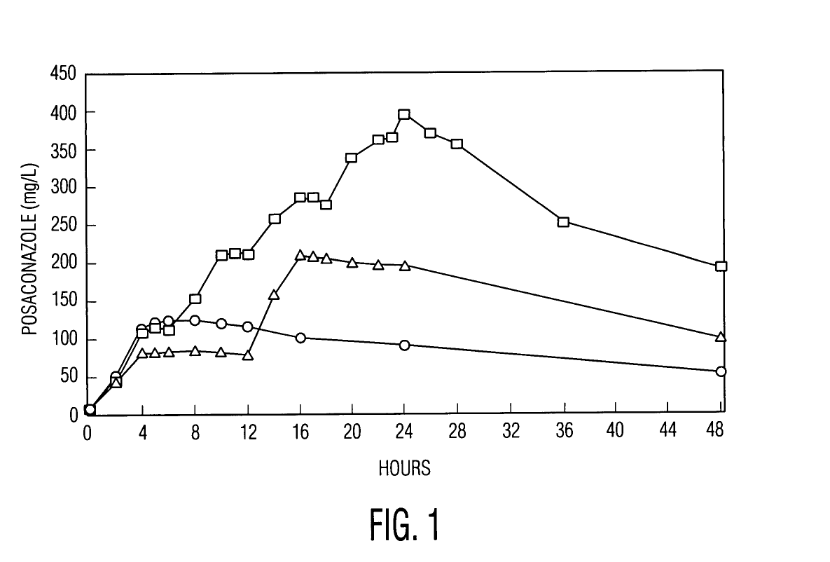

The mean posaconazole plasma concentration-time curves for dosage regimens A, (-●-), B (-Δ-), and C (-□-) are shown in FIG. 1. Mean Cₘₐₓ values (Standard Deviation,“SD”) were 137 (+/−90 ), 225 (+/−115), and 405 (+/−280) ng/mL for regimens A, B, and C, respectively. Mean Tₘₐₓ values (SD) occurred at 7.8 (+/−4.7), 16.6 (+/−3.9), and 24.2 (+/−3.2) hours after the initial dose (corresponding to 7.8, 4.6, and 6.2 hours after the final dose) of each regimen, respectively. Forty-eight hours after administration of the initial dose, mean Cₘᵢₙ concentrations (SD) were 50 (+/−26), 96 (+/−46) and 189 (+/−135) ng/mL for each regimen, respectively.

Pharmacokinetic Parameter Estimates Based on the One-compartment Model

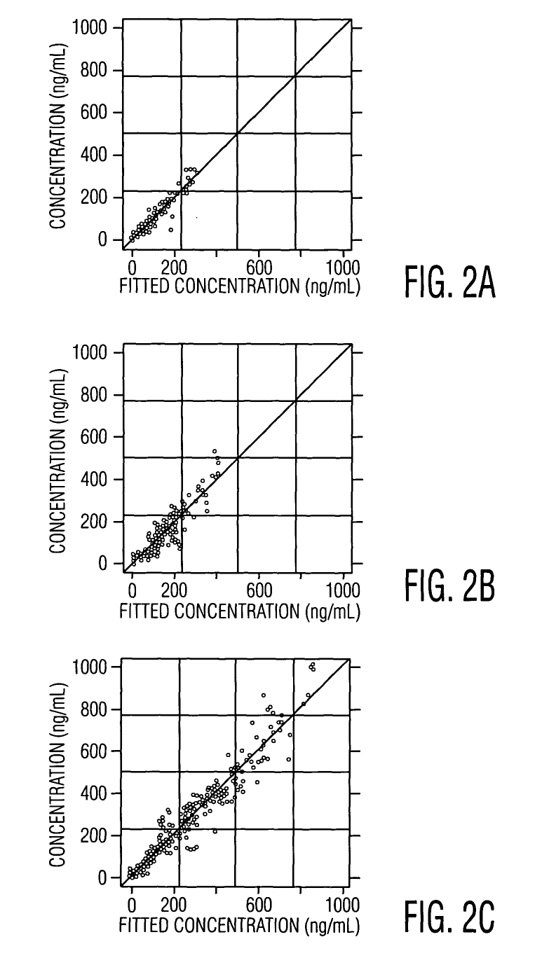

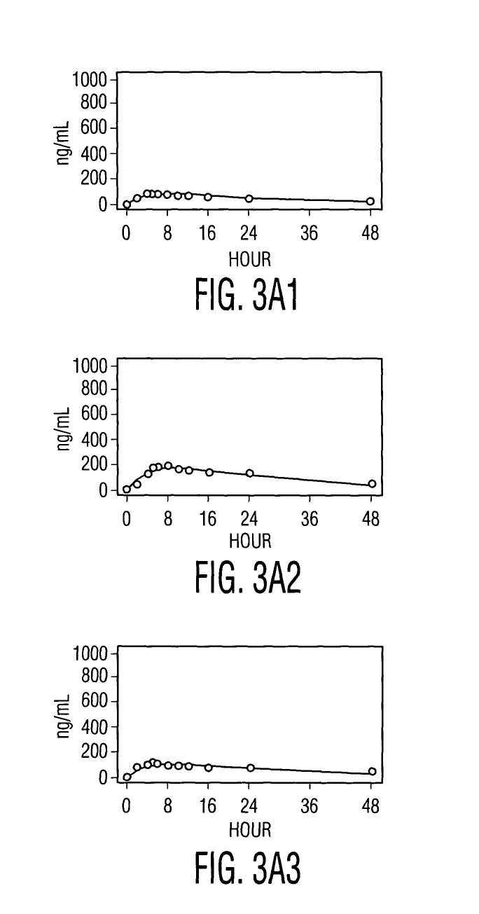

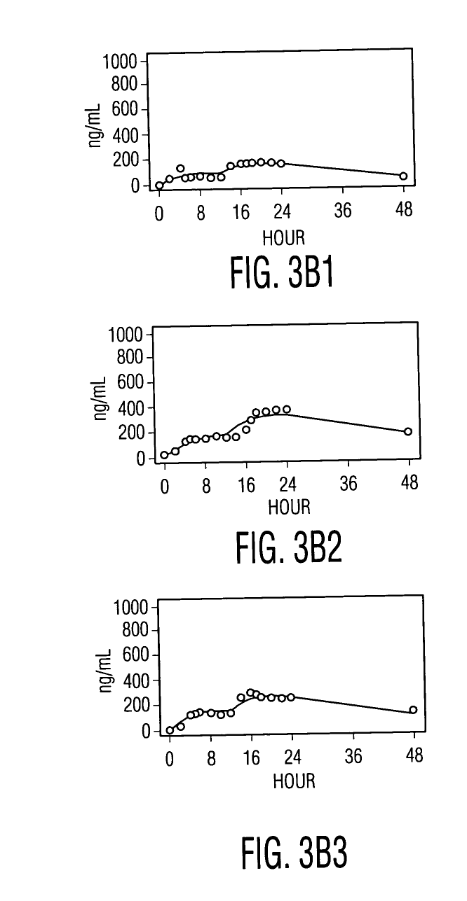

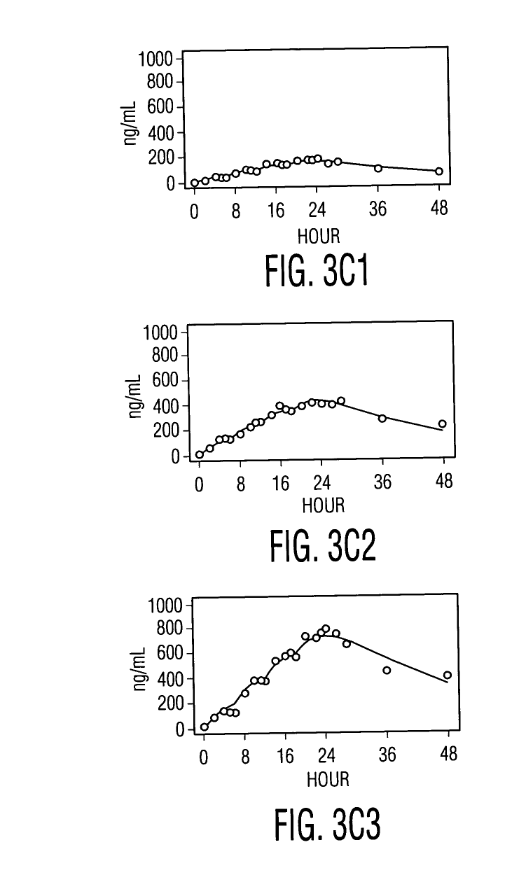

A one-compartment oral model was found to adequately characterize the posaconazole concentrations following all three dosage regimens (Table 1, FIGS. 2A, 2B & 2C). Graphs of the fitted posaconazole concentration profiles (ng/mL) versus observed plasma concentrations (ng/mL) for 3 representative subjects for the 3 dosing regimens are shown in FIGS. 3A1-3, 3B1-3 and 3C1-3. FIGS. 3A1, 3B and 3C are for a first subject; FIGS. 3A2, 3B2, and 3C2 are for a second subject; and FIGS. 3A3, 3B3, and 3C2 are for a third subject. Under fasting conditions, the absorption rate constant was estimated to be 0.197 hour⁻¹, yielding an estimated absorption half-life of 3.5 hours. The elimination rate constant was estimated to be 0.045 hour⁻¹, giving an estimated terminal elimination half-life of 15 hours (Table 1).]

Relative oral bioavailability was significantly different between dosing regimens (P<0.001). Compared with regimen A, the bioavailability fractions (mean±SE) for regimens B and C were estimated to be 1.98±0.35 and 3.20±0.69, respectively. This corresponds to an increase in bioavailability of 98% when posaconazole is given in 2 divided doses versus a single dose, and 220% when given in 4 divided doses versus a single dose. Intersubject coefficients of variation (ISCV) for the bioavailability fractions were 52%, 49%, and 73% for regimens A, B, and C, respectively. ISCV values for the absorption rate constant were 18%, 60%, and 70%, respectively (Table 1). No significant intersubject variability was detected in the elimination rate constant.

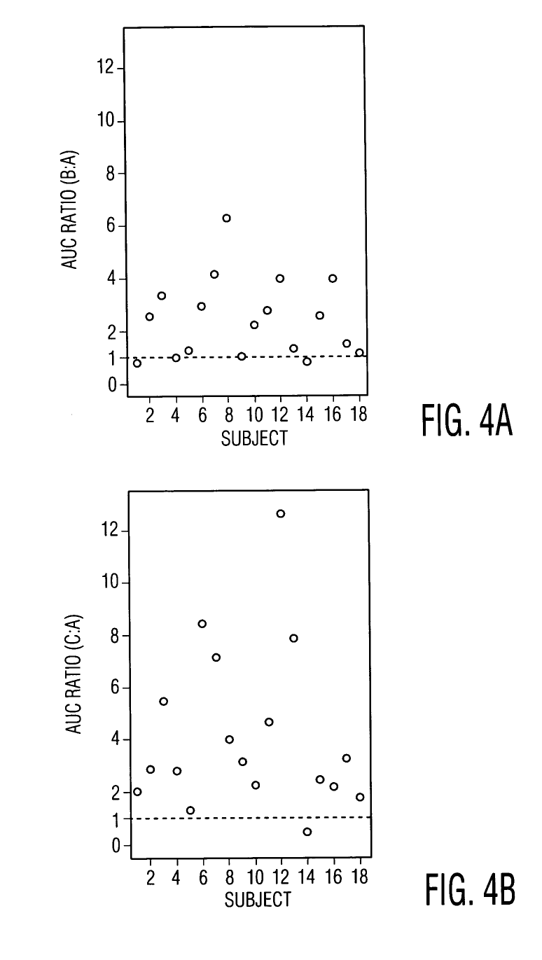

As a result of increased absorption with divided doses, estimated AUC(I) values doubled and tripled with regimens B and C, respectively. Ratios of individual subject estimates of AUC(I) for regimens B and C relative to A were almost all greater than 1, indicating an increase in AUC(I) values following splitting of the administered dose (FIGS. 4A and 4B). Assuming that the model was predictive upon multiple dosing, estimates of AUC over a 24-hour period yielded steady-state AUC(0-24 hr) values of 3,900, 7,700, and 12,400 ng·hr/mL, with estimated steady-state average concentrations of 162, 320, and 517 ng/mL for regimens A, B, and C, respectively.

Safety

Regardless of the dosing regimen, a total daily dose of posaconazole 800 mg was safe and well tolerated. Six (33%) subjects reported at least 1 adverse event, but all adverse events were mild intensity. Two subjects (11%) reported headache with regimens A and B; two subjects (11%) reported chest pain with regimen C. No other individual adverse event was reported by more than 1 subject for any dosing regimen. Vital signs were normal throughout the study and no clinically significant changes in physical examinations, laboratory values, or ECGs were noted.

Conclusions:

The oral bioavailability of posaconazole was calculated to be significantly different between regimens (P value<0.001) increasing with the numbers of doses: regimen B (400 mg BID)/regimen A (800 mg QD)=1.98±0.35 or a 98% increase in posaconazole oral bioavailability for B over A, and regimen C (200 mg QID)/regimen A (800 mgQD)=3.2±0.7, or a 220% increase in posaconazole oral bioavailability for C over A.

The average posaconazole plasma concentrations achieved by orally administering 200 mg of posaconazole using a BID(regimen B) and QID(regimen C) dosing regimen exceed the majority of the Minimum Inhibitory Concentration needed to kill 90% (MIC₉₀) of the clinically relevant pathogenic fungi.

DISCUSSION

The above study demonstrated that posaconazole was orally absorbed in fasted subjects, and exposure to posaconazole increased by dividing the daily dose into 2 doses given twice a day, preferably 12 hours apart or 4 doses given four times a day, preferably 6 hours apart. An 800-mg dose of posaconazole given as 400 mg every 12 hours resulted in a 98% increase in relative oral bioavailability compared to when given as a single dose. Furthermore, a 220% increase in relative oral bioavailability occurred when splitting the 800 mg dose into 4 daily doses. Splitting the daily dose under fasted conditions in accordance with the present invention produces similar posaconazole exposure as when posaconazole is taken with a nonfat meal.

A possible explanation for the increased exposure of posaconazole by splitting the daily dose is a saturation of absorption. The concentration-time curves in FIG. 1 suggest that posaconazole oral absorption is saturable at 200 mg in fasted subjects and therefore administering 200 mg multiple times enhances the total exposure to posaconazole. Saturable absorption was also seen in a previous rising single dose study of posaconazole given with food. In this study, absorption increased in a dose proportional manner and saturated at doses above 800 mg. Therefore, the 800-mg dose was chosen to determine if this maximal exposure could be enhanced using a divided dosing regimen.

Regardless of the dosing regimen, posaconazole was well tolerated. No adverse event was reported by more than 2 patients during any dosing regimen, and all adverse events were mild in intensity. ECGs, laboratory tests, and vital signs were unchanged relative to baseline.

The pharmacokinetic model showed that the major factor influencing exposure of posaconazole was the bioavailability fraction (F). The magnitude of the increase in bioavailability was variable between subjects, thus, allowing the η_{F} to vary with regimen resulted in a substantial improvement in the model fit (Table 2). Inspection of the resulting η_{F1}, η_{F2}, and η_{F3} values, revealed a weak correlation between η_{F1} and η_{F2} (15%) or between η_{F1} and η_{F3} (25%), confirming the importance of the variable η_{F}. This suggests that while the bioavailability increased for most subjects administered regimens B and C, the magnitude of the increase was variable, a phenomenon known as subject by regimen interaction (FIGS. 3A1-3, 3B1-3, and 3C1-3). The rate constants in absorption and in elimination were found not to be different between regimens. However, a significant intersubject variability in the absorption rate constant was detected. No significant intrasubject variability was seen in the elimination rate constant indicating that the variability in posaconazole is a result of absorption and not elimination.

Upon extrapolation of model parameters to multiple dosing, the population mean AUC(0-24 hr) value following oral administration of 200 mg of posaconazole four times a day, preferably q 6 hr was estimated to be 12.4 mcg·hr/mL, representing an average plasma concentration of 0.5 mcg/mL. These estimates, based on data from healthy fasted volunteers, are in close agreement with pharmacokinetic data obtained from a study of patients undergoing high-dose chemotherapy and a bone marrow transplant who were orally administered posaconazole 200 mg q6 h for up to 25 days. In that study, AUC(0-24 hr) at steady state was 10.6 mcg·hr/mL with an average steady-state plasma concentration of 0.428 mcg/mL. Thus, similar exposure of posaconazole in fasted healthy volunteers and in patients at high risk for invasive fungal infections suggests that the split dosing rationale is appropriate. Indeed, dividing posaconazole doses results in plasma concentrations that exceed minimum inhibitory concentrations₉₀ (MIC₉₀) for the majority of pathogenic fungi. These studies combined with other data form the basis for posaconazole dosing regimens in the clinical efficacy trials for posaconazole.

The oral pharmaceutical compositions of the present invention are suitable liquid suspensions containing micronized particles of posaconazole, at least one thickening agent, a non-ionic surfactant and a pharmaceutically acceptable liquid carrier. The mean particle size of posaconazole is at least about 1000 nm, preferably in the range of about 1000 nm to about 2500 nm, or preferably in the range about 1600 nm to about 2200 nm or preferably in the range of about 1200 nm to about 2200 nm or preferably in the range of about 1200 nm to 1800 nm or preferably in the range of 1300 nm to about 1600 nm. Posaconazole used in the oral pharmaceutical composition of the present invention is available from Schering Corporation, Kenilworth, N.J. and may be prepared according to Examples 24 and 32 of U.S. Pat. No. 5,661,151. Suitable non-ionic surfactants include a sorbitan ester of a C₁₀ to C₂₀ acid and is preferably a sorbitan ester of a fatty acid ester such as sorbitan monolaurate, sorbitan monoleate, sorbitan sesquioleate, sorbitan trioleate, sorbitan, monopalmitate, sorbitan monostearate and sorbitan tristearate, or mixtures thereof. The oral pharmaceutical compositions useful in the present invention also contain at least one thickening agent and preferably contain a mixture of two thickening agents including xanthan gum, liquid sugars, starches and celluloses. Preferably the oral pharmaceutical compositions contain a mixture of about 1 mg/mL to about 5 mg/mL of xanthan gum and about 200 mg/mL to about 500 mg/mL, preferably about 350 mg/mL of a liquid sugar such as liquid glucose is used. The oral pharmaceutical compositions of posaconazole also contain a buffer system which maintains the pH of the liquid suspension in the range of about 4 to about 6.0, preferably about 4.5 to about 5.0. Suitable buffer systems include sodium citrate and citric acid. The oral pharmaceutical compositions also may contain an antifoaming agent such as dimethicone or simethicone, water soluble preservatives such as a sodium benzoate or benzalkonium chloride, opacifier agents such as a pharmaceutically acceptable metal oxide such as titanium dioxide and a pharmaceutically acceptable flavoring agent, and a pharmaceutically acceptable liquid carrier such as purified water USD, liquid glucose NF and glycerol, NF, preferably a mixture of purified water USP and liquid glucose, NF. See also International Patent Application No. US 02/10093, filed Apr. 01, 2002, International Publication No. WO 02/080678, published Oct. 17, 2002.

The present invention provides methods of treating or preventing fungal infections in a human of 12 years and older in need of such treating or preventing by orally administering 200 mg of posaconazole four times a day, in single or divided doses, preferably a 200 mg dose of posaconazole four times a day or 400 mg of posaconazole twice a day in single or divided doses, preferably a 2×200 mg of posaconazole two times a day for a time sufficient to eradicate the fungal infection. Determination of the proper dosage and dosage regimen for a particular patient will be determined by the attending clinician in view of the teachings herein and the requirements of the patient, e.g., the patients' food intake, age, severity of the fungal infection and other medications that the patient may be taking. Normally, about 200 mg of oral posaconazole may be administered to the patient four times a day, e.g., every 6 hours until the patient's fungal infection is stabilized in the opinion of the attending clinician; thereafter about 400 mg of oral posaconazole twice a day may be administered until the fungal infection is eradicated for another preferred embodiment. An effective amount of oral posaconazole will be administered to the human in divided doses two, three or four times a day to produce an arithmetic mean steady state average maximum plasma concentration of at least about 300 ng/mL to at least about 520 ng/mL. When about 400 mg of oral posaconazole is administered twice a day (in single or divided doses, the arithmetic mean steady state average maximum plasma concentration (Cₘₐₓ) of at least about 300 ng/mL, preferably at least about 320 20 ng/mL, is produced at a mean time (Tₘₐₓ) after the initial dose in the range of about 12 hours to about 21 hours, preferably about 15 hours to about 19 hours more preferably about 17 hours, and the arithmetic mean area under the concentration-time curve [AUC(0-24 hr)] for posaconazole is about 6,700 ng.hr/mL to about 8,700 ng.hr/mL, preferably about 7,200 ng.hr/mL to about 8,200 ng.hr/mL more preferably about 7,700 ns.hr/mL.

When about 200 mg of oral posaconazole is administered to the human in need of treating or preventing four times a day, preferably every 6 hours, the arithmetic mean steady state average maximum plasma concentration (Cₘₐₓ) of at least about 500 to about 550 ng/mL, preferably about 520 ng/mL of posaconazole is produced at a mean time (Tₘₐₓ) after the initial dose in the range of about 20 hours to 30 hours, preferably about 21.0 hours to 28 hours or more preferably about 24 hours, and the arithmetic mean AUC(0-24 hr) for posaconazole is about 11,400 ng.hr/mL to about 13.400 ng.hr/mL, preferably about 12000 ng.hr/mL to about 13,000 ng.hr/mL, or more preferably about 12,400 ng.hr/mL.

TABLE 1
Pharmacokinetic Parameter Estimates for Each Regimen Based on
a One-Compartment Model

| | | | Intersubject | |
|---|---|---|---|---|
| | | | Coefficient | Increase in |
| | Estimate | | of Variance | Bioavailability |
| Parameter | (SE) | | (%) | vs Regimen A |
| Absorption constant, | 0.197 | (0.02) | | |
| Ka (hr⁻¹) | | | | |
| Regimen A (800 mg | | | 18 | |
| single dose) | | | 60 | |
| Regimen B(400 | | | 70 | |
| mg q12 h) | | | | |
| Regimen C(200 mg q6 h) | | | | |
| Elimination constant, | 0.045 | | | |
| Ke (hr⁻¹) | (0.0032) | | | |
| V/F₁ (L) | 4578 | (630) | | |
| F₁ (Regimen A) | 1.0 | (fixed) | 52 | — |
| F₂ (Regimen B) | 1.98 | (0.35) | 49 | 98% |
| F₃ (Regimen C) | 3.20 | (0.69) | 73 | 220% |

TABLE 2
A Statistical Comparison of Each Nested Model

| | | Log | | Model | | |
|---|---|---|---|---|---|---|
| Model | Parameters | Likelihood | d.f. | Comparison | Δ¹ | P-value |
| I (F unchanged with regimen) | Ka, Ke, V, F, η_{Ka}, η_{F} | −5415 | 6 | — | | |
| II (F varies with regimen) | Ka, Ke, V, F˜R^{b}, η_{Ka}, η_{F} | −4908 | 8 | II vs I | 1014 | <0.0001 |
| II (F and η_{F} varies with regimen) | Ka, Ke, V, F˜R, η_{Ka}, η_{F}˜R | −4580 | 10 | III vs II | 665 | <0.0001 |
| IV (F, η_{F} and η_{Ka} varies with regimen) | Ka, Ke, V, F˜R, η_{Ka}˜R, η_{F}˜R | −4558 | 12 | V vs IV | 43 | <0.0001 |

¹Likelihood ratio

What is claimed is:

1. A method of treating or preventing fungal infections in humans of 12 years and older in need of such treating or preventing which comprises orally administering an effective amount of posaconazole in divided doses two to four times a day to produce an arithmetic mean steady state average maximum plasma concentration of posaconazole of at least about 300 ng/mL to at least about 520 ng/mL

2. The method of claim 1 wherein the arithmetic mean steady state maximum plasma concentration (Cₘₐₓ) of posaconazole of at least about 300 ng/mL to about 320 ng/mL is produced at a mean time(Tₘₐₓ) after the initial dose in the range of about 12 hours to about 21 hours.

3. The method of claim 1 wherein the arithmetic mean steady state maximum plasma concentration (Cₘₐₓ) of at least about 500 ng/mL to about 520 ng/mL of posaconazole is produced at an arithmetic mean Tₘₐₓ of about 21 hours to about 28 hours.

4. The method of claim 1 wherein a plot of the plasma concentration of posaconazole over time yields an arithmetic mean AUC(0-24hr) for posaconazole in the range of about 7,700 ng.hr/mL to about 12,400 ng.hr/mL.

5. The method of claim 1 wherein the fungal infection in need of treating and/or preventing is an invasive fungal infection.

6. The method of claim 1 wherein the humans in need of treating or preventing are immuno-compromised patients.

7. The method of claim 1 wherein the humans in need of treating or preventing are neutropenic patients.

8. The method of claim 1 wherein the humans in need of treating or preventing are fasted patients.

9. The method of claim 1 wherein the amount of posaconazole administered is about 200 mg four times a day.

10. The method of claim 1 wherein the amount of posaconazole administered is about 400 mg two times a day.

11. A method of treating or preventing a fungal infection in humans of 12 years and older in need of such treating or preventing which comprises orally administering an effective amount of posaconazole in divided doses two to four times a day to produce an arithmetic mean steady state minimum plasma concentration (Cₘᵢₙ) of posaconazole of at least about 50 ng/mL at about 48 hours after the initial dose of the effective amount of posaconazole.

12. The method of claim 11 wherein the human in need of treating or preventing is an immuno-compromised patient.

13. The method of claim 11 wherein the human in need of treating or preventing is a neutropenic patient.

14. The method of claim 11 wherein the human in need of treating or preventing is a fasted patient.

15. A method treating or preventing fungal infections in humans of 12 years and older in need of such treating or preventing which comprises orally administering about 200 mg of posaconazole four times a day to produce an arithmetic mean steady state average maximum plasma concentration of posaconazole of at least about 500 ng/mL.

16. The method of claim 15 wherein the human in need of treating and/or preventing is an immuno-compromised patient.

17. The method of claim 15 wherein the human in need of treating and/or preventing is a neutropenic patient.

18. The method of claim 15 wherein the human in need of treating and/or preventing is a fasted patient.

19. A method treating or preventing fungal infections in a human of 12 years and older in need of such treating and /or preventing which comprises orally administering about 200 mg of posaconazole four times a day to produce a population mean area under the concentration-time (0-24hr) curve of of posaconazole of about 12 mcg.hr/mL.

20. The method of claim 19 wherein the human in need of treating and/or preventing is an immuno-compromised patient.

21. The method of claim 19 wherein the human in need of treating and/or preventing is a neutropenic patient.

22. The method of claim 19 wherein the human in need of treating and/or preventing is a fasted patient.

23. A method of treating or preventing fungal infections in humans of 12 years and older in need of such treating or preventing which comprises orally administering an effective amount of posaconazole in divided doses two to four times a day to produce an arithmetic mean steady state average maximum plasma concentration of posaconazole that exceeds the majority of the Minimium Inhibitory Concentrations needed to kill 90% (“MICs₉₀”) of the clinically relevant pathogenic fungi.

24. A method treating or preventing fungal infections in a human of 12 years and older in need of such treating and /or preventing which comprises orally administering about 200 mg of posaconazole four times a day to produce an average plasma concentration of posaconazole of about 0.5 mcg.hr/mL.

25. The method of claim 24 wherein the human in need of treating or preventing is an immuno-compromised patient.

26. The method of claim 24 wherein the human in need of treating or preventing is a neutropenic patient.

27. The method of claim 24 wherein the human in need of treating or preventing is a fasted patient.

28. A method of treating or preventing a fungal infection in a human of 12 years of age and older in need of such treating or preventing which comprises orally administering to such a human about 200 mg of posaconazole four times a day until the patent is considered stable by an attending clinician and thereafter orally administering about 400 mg of posaconazole twice a day until the fungal infection is eradicated as shown by standard antifungal testing procedures.

29. A method of treating or preventing fungal infections in a human of 12 years of age and older in need of such treating or preventing which comprises orally administering to such a human about 200 mg of posaconazole four times a day.

30. A method treating or preventing fungal infections in a human of 12 years of age and older in need of such treating or preventing which comprises orally administering to such a human a total dose of about 800 mg of posaconazole a day in three divided doses.

31. A method treating or preventing fungal infections in a human of 12 years of age and older in need of such treating or preventing which comprises orally administering to such a human about 400 mg of posaconazole two times a day.
